(19) United States
(12) Patent Application Publication
(10) Pub. No.: US 2018/0325691 A1
(43) Pub. Date: Nov. 15, 2018

(54) METHODS, IMPLANTS, AND TOOLS FOR FUSION OF SCOLIOTIC SPINES

(71) Applicant: Sharad Rajpal, Boulder, CO (US)

(72) Inventor: Sharad Rajpal, Boulder, CO (US)

(21) Appl. No.: 15/978,352

(22) Filed: May 14, 2018

Related U.S. Application Data
(60) Provisional application No. 62/506,110, filed on May 15, 2017.

(51) Int. Cl. A61F 2/44

(52) U.S. Cl. CPC A61F 2/4425; A61F 2002/30538; A61F 2002/4435

(57) ABSTRACT

An implant for use in correcting scoliosis and fusing a spine includes a top plate; a bottom plate; and a height adjustment mechanism configured to permit independent adjustment of a rear height between top plate and bottom plate, and a front height between top plate and bottom plate. The front and bottom plate of the implant form a wedge of adjustable angle and height. A method of fusing a patient's spine using the implant includes approaching the spine laterally; removing at least one intervertebral disk; inserting the implant; and adjusting the implant to form a wedge of a desired angle and height. In embodiments, the adjustment mechanism includes slideable wedges; in other embodiments the adjustment mechanism includes threaded gears engaging threaded studs to adjust a length that determines height of a portion of the implant.

### CROSS-REFERENCE TO RELATED APPLICATIONS

This application claims priority to U.S. Provisional Patent Application Ser. No. 62/506,110, filed May 15, 2017, entitled “METHODS, IMPLANTS, AND TOOLS FOR FUSION OF SCOLIOTIC SPINES”, which is incorporated herein by reference.

### BACKGROUND

Scoliosis is an abnormal, curvature of the spine. While mild scoliosis may be treated by bracing, surgical treatments are often used to treat more severe scoliosis. Scoliosis may be treated by fusion surgery. When surgical fusion treatment is used, typical spinal fusion surgery uses either a posterior, lateral, or anterior approach to vertebrae (FIG. 1) of the spine. Each vertebra 100 typically has a body 102, a mating surface 104 for an intervertebral disk, a pair of pedicles 106 connecting the body 102 to paired transverse processes 108, and a pair of laminae 110 connecting the transverse processes to a single spinous process 112. The laminae 110, pedicles 106, and body 102 surround a neural canal 114. When a fusion is performed, the aim is to replace the fibrous disk that is normally located between the bodies of two vertebrae with fusion devices, then allow bone to grow into and through the fusion devices to form a solid connection between the two vertebral bodies. Surgical access to the vertebral bodies is normally done either with an anterior or lateral approach, requiring temporary displacement of abdominal or thoracic organs, or with a posterior approach that poses risk to nerves and spinal cord.

Scoliosis itself is an abnormal curvature of the spine. A vertical cross section of a normal vertebral body 148 is rectangular. When scoliosis has been present for a long time, some or all vertebrae, such as vertebra 150, 152 (FIG. 2) may have bodies that are no longer rectangular in cross section, with cross sections that are narrower on one side 154 than on another side 156.

### SUMMARY

An implant for use in correcting scoliosis and fusing a spine includes a top plate; a bottom plate; and a height adjustment mechanism configured to permit independent adjustment of a rear height between top plate and bottom plate, and a front height between top plate and bottom plate. The front and bottom plate of the implant form a wedge of adjustable angle and height. A method of fusing a patient's spine using the implant includes approaching the spine laterally; removing at least one intervertebral disk; inserting the implant; and adjusting the implant to form a wedge of a desired angle and height. In embodiments, the adjustment mechanism includes slideable wedges, in other embodiments the adjustment mechanism comprises a plurality of threaded gears

### BRIEF DESCRIPTION OF THE FIGURES

FIG. 1 is a top view of a vertebra showing body, processes, and spinal canal.

FIG. 2 is a schematic anterior view of vertebral bodies in scoliosis, showing restructured bone shapes.

FIG. 3 illustrates a lateral view of top plate of an implant for correction of scoliosis during vertebral fusion.

FIG. 3A is a bottom view of the top plate of the implant of FIG. 3.

FIG. 4 illustrates a distal and near slider of an implant for correction of scoliosis during vertebral fusion.

FIG. 5 illustrates a lateral view of the sliders of FIG. 4.

FIG. 6 is a top plan view of a bottom plate of the implant having wedge surfaces for engaging the sliders of FIG. 4.

FIG. 7 is a lateral view of the bottom plate of FIG. 6.

FIG. 8 is a lateral view of an assembled implant incorporating the top plate of FIG. 3-3A, slider of FIG. 4-5, and bottom plate of FIG. 6-7.

FIG. 9 is a lateral view of the assembled implant of FIG. 8 with slider wedges positioned to give a wedge shape to the implant with the rear more fully expanded than the front.

FIG. 10 is a lateral view of the assembled implant of FIG. 8 with sliders positioned to give even expansion on both ends of the implant.

FIG. 11 is a flowchart of a surgical procedure using the implant of FIG. 3-10.

FIG. 12 is a schematic anterior view of vertebral bodies in scoliosis with implants according to FIG. 3-10, showing restructured bone shapes and wedged implants, and substantially less overall curvature than the pre-surgery spine of FIG. 2.

FIG. 13 is a schematic top view of a bottom plate showing four threaded gears.

FIG. 14 is a schematic bottom view of a top plate showing four threaded studs.

FIG. 15 is a schematic side view of the top and bottom plates of FIGS. 13 and 14 with even expansion between front and back gears.

FIG. 16 is a schematic side view of an implant comprising the top and bottom plates of FIGS. 13 and 14 adjusted for greater expansion at the rear of the implant than at front of the implant.

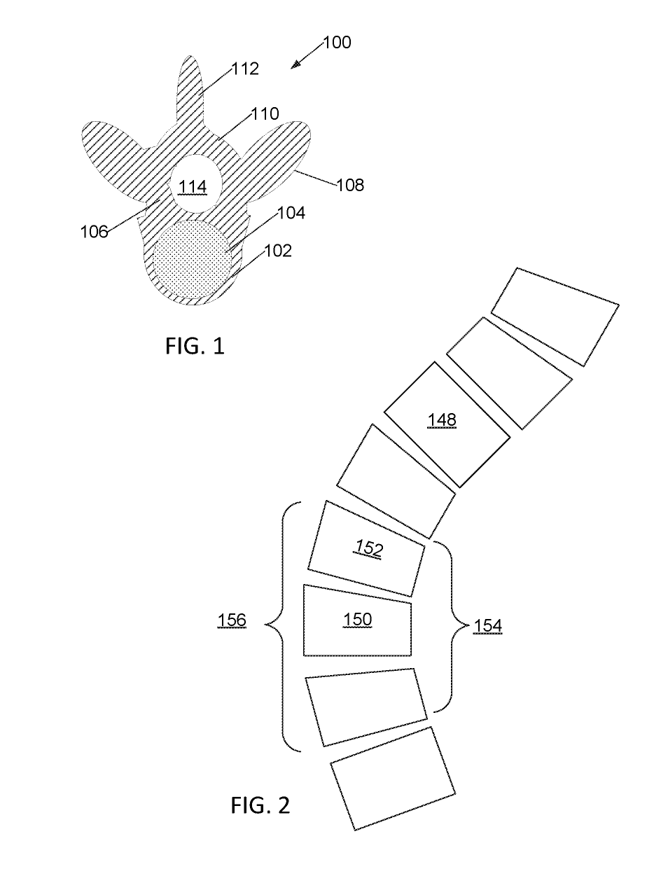

### DETAILED DESCRIPTION OF THE EMBODIMENTS

We believe that an improved fusion surgery for scoliotic spines is one with lateral, rather than an anterior or posterior approach because fewer critical structures must be displaced to gain access to the vertebral bodies. Further, we believe that a wedge-shaped implant with independently-adjustable height at each of a forward and rear end of the implant offers advantages during such surgery because the wedge can be individually adjusted to optimize correction for each patient. Although there are wedge-shaped implants on the market, those implants can only be used with the wide side to the incision side of the implant, and those implants do not provide for adjustability to more than two possible implant heights or wedge angles.

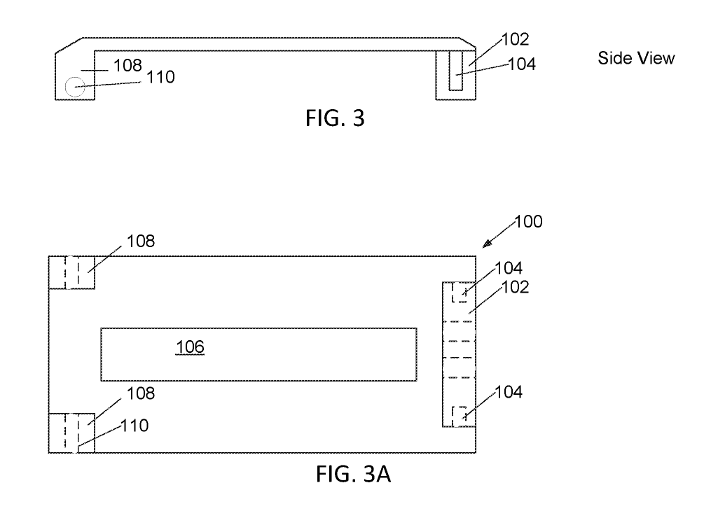

An implant has a top plate 100 (FIG. 3, 3A) having a ridge 102 with slots 104 adapted to slideably engage a pin 702 (FIG. 7) engaged in an ear 704 of a bottom plate 700. The top plate 100 has a roughened titanium top surface adapted for bone ingrowth and an opening 106 permitting bone growth into cavities of the implant. In alternative embodiments, the top 100 and bottom 700 plates have a hydroxyapatite coating to encourage bonding and ingrowth of bone. The top plate also has a pair of ears 108 each having a threaded hole 110 adapted to engaging a threaded pin (not shown) for engaging a slot 706 in an ear 708 of bottom plate 700. Top plate 100 has chamfered upper ends to permit easier insertion than would otherwise be the case.

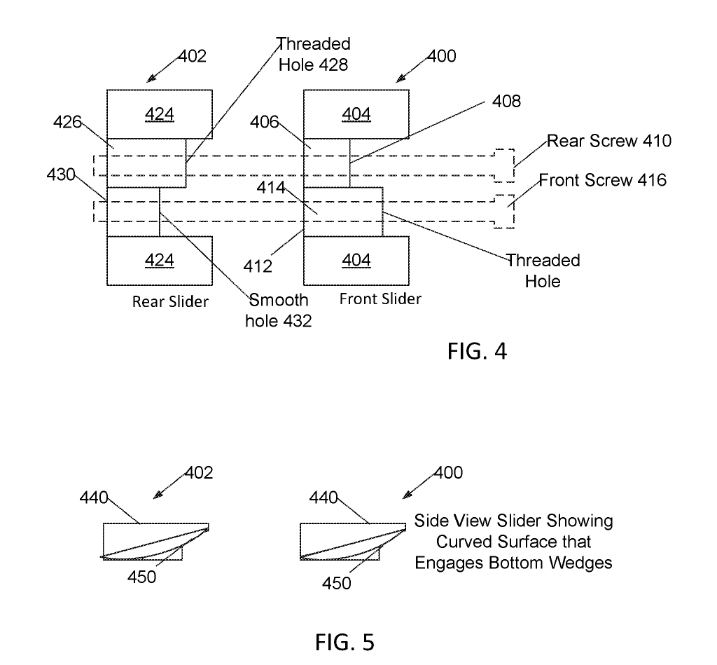

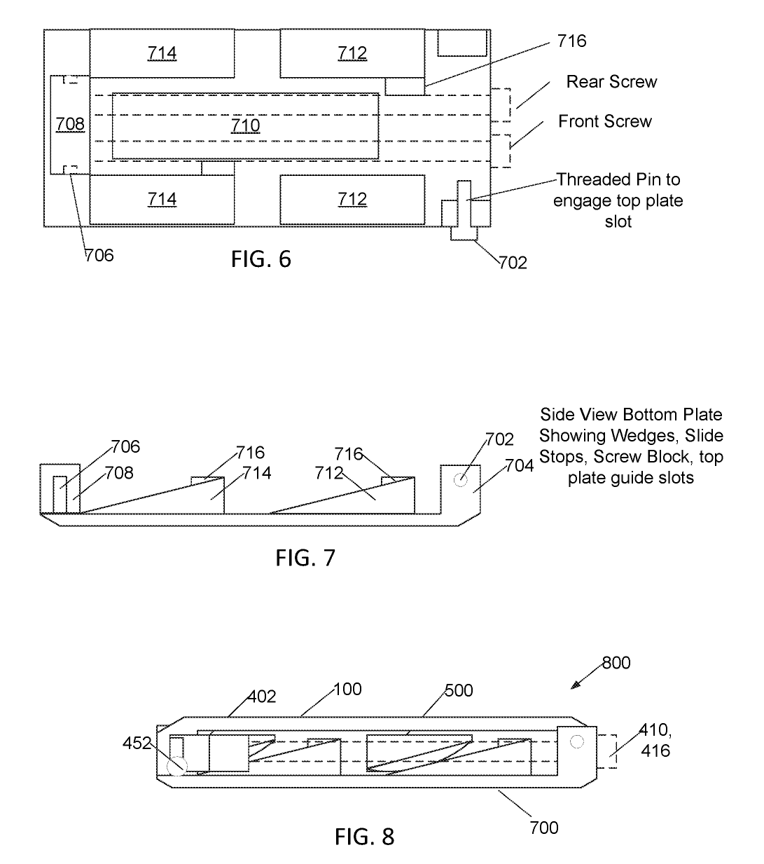

Bottom plate 700 has a bottom surface of roughened titanium adapted for bone ingrowth and an opening 710 (FIG. 6) permitting bone growth into cavities of the implant. Bottom plate 700 also has front wedges 712 and rear wedges 714 configured for height adjustment with the sliders 400, 402 illustrated in FIGS. 4 and 5. The bottom plate 700 may also have one or more adjustment stop blocks 716 configured to limit movement of the sliders 400, 402. Bottom plate 700 has chamfered lower ends to permit insertion.

Sliders 400, 402 are illustrated in more detail in FIGS. 4 and 5. Front slider 400 has two wedge portions 404, an interconnecting portion 406 having a smooth, non-threaded-hole 408 large enough to pass a rear screw 410, and a second interconnecting portion 412 having a threaded hole 414 adapted to engage threads of a front screw 416. Similarly, rear slider 402 has two wedge portions 424, a first interconnecting portion 426 having a threaded hole 428 adapted to engage threads of the rear screw 410, and a second interconnecting portion 430 having a smooth hole 432.

Viewed from the side, each slider 400, 402 has a flat surface 440 (FIG. 5) adapted to engage a bottom surface of top plate 100 and an arcuate surface 450 of wedge portions 404 adapted to engage wedge portions 712, 714 of bottom plate 700. In a particular embodiment the flat surface 440 and the mating bottom surface of top plate 100 are engraved with a fishscale pattern to resist sliding of these surfaces against each other inhibiting movement of the sliders in the direction of the rear tabs 108. In another particular embodiment, arcuate surface 450 of wedge portions 404 and their mating surfaces of wedge portions 712, 714 are engraved with a fishscale pattern to resist sliding of these surfaces against each other, inhibiting movement of the sliders towards the rear ear 708 of the bottom plate.

The top plate, bottom plate, front, and rear sliders are assembled with front and rear screws 410, 416 and four threaded pins 452 into an implant 800 as illustrated in FIG. 8; FIG. 8 shows the front and rear sliders to the rear of their travel with the rear and front of the implant. Threaded pins 452 engage slots of top 100 and bottom 700 plates to permit expansion of the implant in a vertical direction without allowing relative motion of the top and bottom plates in a horizontal direction. Front and rear screws 410, 416 have heads that engage against ridge 102 of top plate 100 so that when tightened they draw front 400 and rear 402 sliders respectively towards ridge 102 and force wedge portions 404 of the sliders against wedge portions 712, 714 to drive expansion of the implant in a vertical direction.

In an embodiment, front and rear screws 410, 416 extend through slots in bottom plate 700 ear 708 and a nut, the nut staked, welded, or soldered in place on screw 410, 416, to permit rotation of the screws to push front and rear sliders 400, 402 towards ear 708 and retract expansion of the implant. In an alternative embodiment, similar operation is achieved with a nut staked in place on screws 410, 416 directly to a rear of ridge 102 for the same purpose.

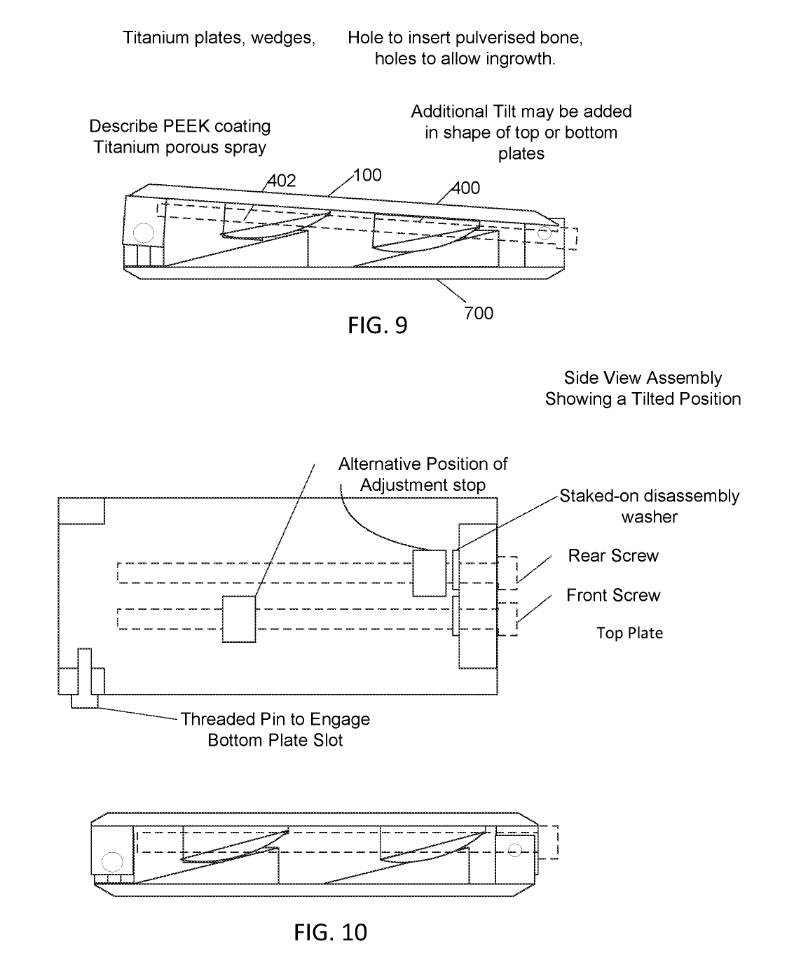

FIG. 9 illustrates the assembled implant of FIG. 8 where the sliders 402, 400 have been positioned other than uniformly, causing the implant to take a wedge shape. The sliders illustrated are formed as wedges with arcuate surfaces. In this illustration, the rear slider 402 has been drawn by the rear screw towards the ridge 102 of the top plate. The sliders each engage one or more fixed wedges, the fixed wedges may in an embodiment be formed as part of the top or bottom plate (bottom plate illustrated), and in alternative embodiments the fixed wedges are formed separately and permanently attached to the top or bottom plate. In alternative embodiments, the fixed wedges are formed with arcuate surfaces engaging with planar wedge surfaces on the sliders.

Vertical height adjustment of each end of the implant is independent, but is limited. While top plate 100 and bottom plate 700 are shown as essentially flat with parallel upper and lower surfaces, either or both top 100 and bottom 700 plate may be fabricated with a wedge-shaped cross section. In this way, an embodiment may have an adjustment range from zero to plus or minus 15 degrees of wedge angle, and another embodiment may have an adjustment range from plus 10 to plus 30 degrees of wedge angle, as appropriate for patients. Similarly, the implants may be fabricated in multiple versions each having a different unexpanded heights for use in a variety of patients.

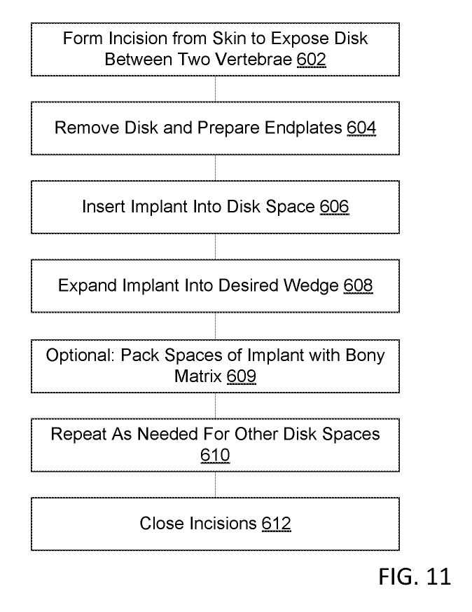

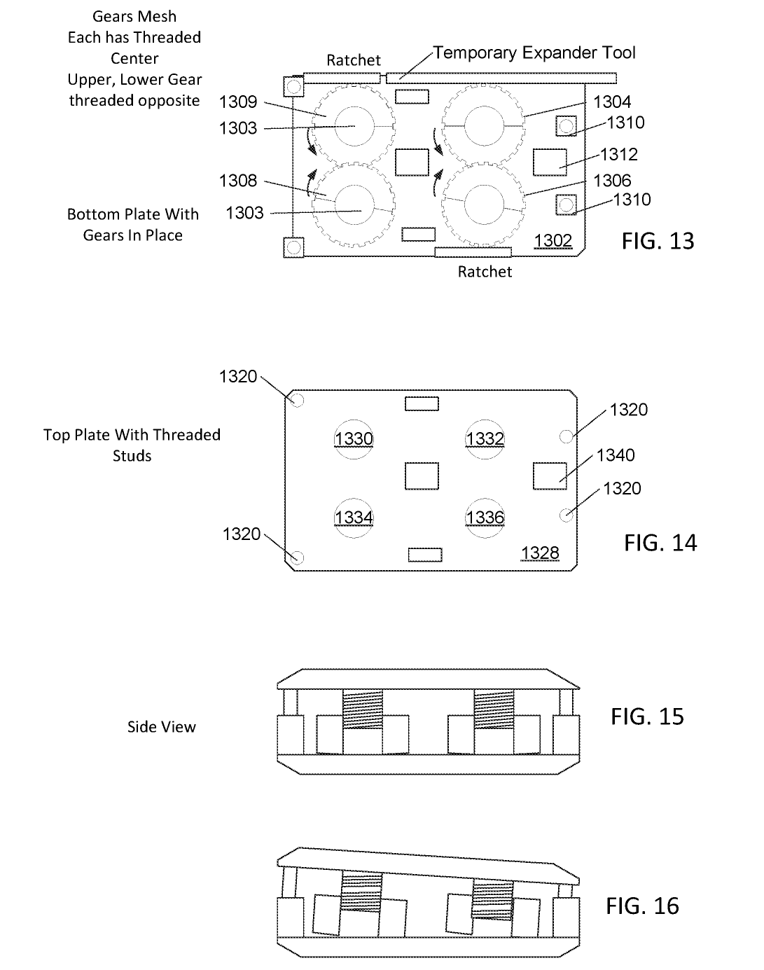

An approximate flowchart of a method of using the implants herein described to correct scoliosis is illustrated in FIG. 11. After exposing 602 the disk between two vertebrae, the surgeon removes 604 the disk leaving an intervertebral space and prepares the bones by roughening endplates and removing articular cartilage. Implants are manufactured in an assortment of sizes, these are stocked at surgical facilities. The surgeon selects an appropriate implant from the assortment of sizes of implants that, when adjusted to minimum height, will fit into the intervertebral space and has a desired range of expansion in height and angle, and inserts 606 the implant into the intervertebral space; the surgeon then uses an adjusting tool to engage adjusting screws (for the implant of FIGS. 3-10) or ratchet (for the implant of FIGS. 13-16) and adjusts 608 front and rear implant height independently to form the implant into the desired wedge shape. In embodiments windows and other openings are provided wherever possible, such as in portions of the top and bottom plate that do not interact directly with the sliders, in areas where mechanical function of the implant is not disturbed, to permit bony ingrowth. These windows and other openings may optionally be packed 609 with autograft or heterograft bony matrix material to encourage bony ingrowth and fusion. Steps of exposing 602, removing 604, selecting, inserting 606, adjusting 608, and packing are repeated with additional implants at additional vertebrae as necessary for additional disk spaces.

In some cases, scoliosis involves curves in opposite directions, requiring some implants with opposite wedge orientations. An advantage of the implants described herein is that they can provide opposite wedge orientation using a lateral approach from a same side of the patient—reducing trauma and expediting healing.

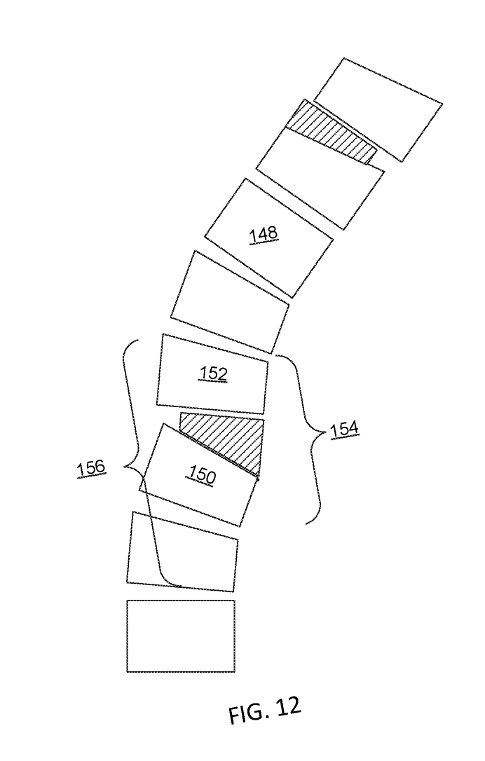

FIG. 12 is a schematic anterior view of vertebral bodies in scoliosis with implants according to FIG. 3-10, showing restructured bone shapes and wedged implants, and substantially less overall curvature than the pre-surgery spine of FIG. 2.

FIG. 13 is a schematic top view of a bottom plate 1300 showing four threaded gears that rest on the bottom plate 1300. The threading of each gear is in an axial hole 1303 of each gear. These threaded gears 1304, 1306, 1309, 1308 mesh in pairs, a front pair 1304, 1306 and a rear pair 1309, 1308. The threaded gears of each pair have opposite threads, in a particular embodiment a first gear of front pair 1304 has a left-hand thread, while second gear of front pair 1306 has a right-hand thread, and a first gear of rear pair 1309 has a left-hand thread, while second gear of front pair 1308 has a right-hand thread. Bottom plate may have holes 1312 to permit ingrowth of bone and to permit grasping the plate with a tool for removal from the patient. Bottom plate 1300 also has four studs 1310, each with a hole for an alignment pin 1320 of the top plate 1322 (FIG. 14). The gears of the front pair do not mesh with gears of the rear pair. A ratcheting mechanism is provided to rotate gears of the front pair when pressed by a tool, and another ratcheting mechanism is provided to rotate gears of the rear pair when pressed by the tool. The gears of each pair have a convex curved surface that engages the bottom plate, and a flat surface on a top plate side.

In the embodiment of FIG. 13-16, the studs are components with male threads and the gears as being components with female threads, and height adjustment is performed by rotating the components with a female thread relative to the components with a male thread. Each component with male thread is engaged with a component with female thread, so relative rotation of the components changes an overall length of the engaged male and female threaded components, this overall length determines height of a part of the implant and changes in this overall length alters height of that part of the implant.

A top plate 1328 of the implant of FIGS. 13-16 is illustrated in FIG. 14, and has four threaded studs 1330, 1332, 1334, 1336, each with thread matching one of the four gears 1304, 1306, 1309, 1308. The implant is assembled inverted by meshing front pair gears, screwing them onto front studs 1332, 1336, meshing rear pair gears, screwing them onto rear studs 1330, 1334, attaching the ratchets, and placing the bottom plate on the gears with alignment pins 1320 of the top plate entering holes of the alignment studs 1310 of the bottom plate. Top plate may have holes 1340 and may have a hydroxyapatite coating, or a polymer coating with embedded hydroxyapatite and growth factors, for allowing and encouraging ingrowth of bone during the healing process.

In an alternative embodiment of the embodiment of FIGS. 13-16, the ratchets are removed and replaced with a worm gear having a fitting, such as a hex-socket head or Phillips screw head, for attachment of an insertion and adjustment tool.

FIG. 15 is a schematic side view of the top and bottom plates of FIGS. 13 and 14 with even expansion between front and back gears. This is achieved by using the ratcheting mechanisms to rotate the front pair and back pair of gears evenly.

FIG. 16 is a schematic side view of an implant comprising the top and bottom plates of FIGS. 13 and 14 with greater expansion at the rear of the implant than at front of the implant. This is accomplished by using the ratchet mechanisms to rotate the front pair of gears differently than rotating the back pair of gears.

Applicant anticipates that the adjustable implants herein disclosed have been referred to as having a top plate and a bottom plate, and may be implanted in a post-diskectomy interbody space with the top plate adjacent a lower surface of a superior vertebral body and the bottom plate atop an upper surface of an inferior vertebral body. Applicant also anticipates that the adjustable implants herein disclosed may be inverted, implanted in a post-diskectomy interbody space with the bottom plate adjacent a lower surface of a superior vertebral body and the top plate atop an upper surface of an inferior vertebral body.

Applicant anticipates that, in specific embodiments, the implants herein described are fabricated from one or more materials in the group titanium, porous titanium with a hydroxyapatite coating, polyether ether ketone (PEEK) or other thermoplastics in the polyaryletherketone (PAEK) family, PEEK with embedded calcium phosphate or hydroxyapatite particles, stainless steel, cobalt, chromium, cobalt-chromium alloy, and stainless steel. In some embodiments, growth factors and other additives are applied to the implant to encourage bone ingrowth. Further, in embodiments windows and other openings are provided wherever possible without disrupting mechanical function of the implants to permit bony ingrowth, these openings may be packed 609 with autograft or heterograft bony matrix material to encourage bony ingrowth and fusion after the implants have been expanded into their final wedge shape.

Since natural intervertebral disks and vertebral bodies are rounded, not square, it is anticipated that corners of the implants are rounded or chamfered to ease insertion and provide good fit to the surfaces of these bodies that remains after discectomy.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. An implant for use in fusing a spine, the implant configured for insertion into a post-diskectomy intervertebral space between an upper and a lower vertebral body, the implant comprising:
a top plate configured to mate with a lower surface of the upper vertebral body;
a bottom plate configured to mate with an upper surface of the lower vertebral body; and
a height adjustment mechanism configured to permit independent adjustment of a rear height between top plate and bottom plate, and a front height between top plate and bottom plate, such that the implant may be adjusted into a shape wherein the front and bottom plate form a wedge, the wedge being of adjustable angle and height.

2. The implant of claim 1 wherein the adjustment mechanism comprises a plurality of sliders each configured to engage a fixed wedge surface attached to or formed on a plate selected from the top and bottom plate, and height adjustment is performed by sliding at least one of the plurality of sliders relative to the fixed wedge surface.

3. The implant of claim 2 wherein the height adjustment mechanism further comprises a first screw coupled to reposition at least a first slider of the plurality of sliders, and a second screw coupled to reposition at least a second slider of the plurality of sliders, the first and second sliders being different.

4. The implant of claim 3 where both the first and second screw have a head adapted for coupling to an insertion tool on a proximal side of the implant, the first screw and first slider being coupled to adjust height between top and bottom plate near the proximal side of the implant, and the second screw and second slider being coupled to adjust height between top and bottom plate distal to the proximal side of the implant.

5. The implant of claim 4 the top and bottom plate being heightwise slideably engaged.

6. The implant of claim 1, the adjustment mechanism comprising a plurality of threaded male components, each threaded male component engaged with a threaded female component in pairs, the threaded male and threaded female components of each pair configured so relative rotation of the threaded male component and threaded female component adjust a height between top plate and bottom plate of a portion of the implant.

7. The implant of claim 6 wherein the threaded female components comprise threaded gears.

8. The implant of claim 7 wherein a first threaded gear of the threaded gears is engaged with a second threaded gear of the plurality of threaded gears, and wherein the first threaded gear has a left-hand thread and the second threaded gear has a right-hand thread.

9. The implant of claim 8, further comprising a mechanism adapted to couple to an insertion tool and to rotate the first and second threaded gears.

10. The implant of claim 9 wherein a third threaded gear of the threaded gears is engaged with a fourth threaded gear of the plurality of threaded gears, the third threaded gear has a left-hand thread and the fourth threaded gear has a right-hand thread, and the implant configured so adjustment of the first and second threaded gears is independent of adjustment of the third and fourth threaded gears.

11. The implant of claim 10 the top and bottom plate being heightwise slideably engaged.

12. A method of fusing a patient's spine comprising:
approaching the spine laterally;
removing at least one intervertebral disk;
inserting an implant configured to permit independent adjustment of a rear height between a top plate and a bottom plate, and a front height between top plate and bottom plate, such that the front and bottom plate form a wedge of adjustable angle and height; and
adjusting the implant to form a wedge of a desired angle and height.

13. A method of correcting scoliosis comprising the method of claim 12, the implant forming the wedge of a desired angle and height being positioned to correct the scoliosis.

14. The method of claim 13, the implant comprising an adjustment mechanism with a plurality of sliders each configured to engage a fixed wedge surface attached to or formed on a plate selected from the top and bottom plate, and height adjustment is performed by sliding at least one of the plurality of sliders relative to the fixed wedge surface.

15. The method of claim 14 wherein the height adjustment mechanism further comprising a first screw coupled to slide at least a first slider of the plurality of sliders, and a second screw coupled to slide at least a second slider of the plurality of sliders, the first and second sliders being different.

16. The method of claim 12, the adjustment mechanism comprising a plurality of threaded male components, each threaded male component engaged with a threaded female component in pairs, the threaded male and threaded female components of each pair configured so relative rotation of the threaded male component and threaded female component adjust a height of a portion of the implant.

17. The method of claim 16 wherein the threaded female components are threaded gears.

18. The method of claim 17 wherein a first threaded gear of the threaded gears is engaged with a second threaded gear of the plurality of threaded gears, and wherein the first threaded gear has a left-hand thread and the second threaded gear has a right-hand thread.

19. The method of claim 18, further comprising a mechanism adapted to couple to an insertion tool and to rotate the first and second threaded gears.

20. The implant of claim 19 wherein a third threaded gear of the threaded gears is engaged with a fourth threaded gear of the plurality of threaded gears, the third threaded gear has a left-hand thread and the fourth threaded gear has a right-hand thread, and the implant configured so adjustment of the first and second threaded gears is independent of adjustment of the third and fourth threaded gears.
